(19) United States
(12) Patent Application Publication
(10) Pub. No.: US 2003/0118473 A1
(43) Pub. Date: Jun. 26, 2003

(54) Method and apparatus for ozone decontamination of biological liquids

(76) Inventors: Gerard V. Sunnen, New York, NY (US); James R. Robinson, Livermore, CA (US)

Correspondence Address: MORGAN & FINNEGAN, L.L.P., 345 Park Avenue, New York, NY, 10154-0053, US

(21) Appl. No.: 10/285,988

(22) Filed: Nov. 1, 2002

Related U.S. Application Data
(60) Provisional application No. 60/335,735, filed on Nov. 2, 2001.

(51) Int. Cl. A01N 1/00; A61L 2/00

(52) U.S. Cl. 422/28; 422/44; 422/186.07

(57) ABSTRACT

The present invention relates to a delivery system and a treatment protocol designed to accurately and reliably deliver ozone to biological liquids or to other liquids containing reactive solutes or particles. To effect the accurate delivery of ozone to such liquids, ozone is delivered to the liquid via a gas permeable interface, under controlled conditions, while the liquid passes along the opposite side of the interface. In one variant, ozone is interfaced with the liquid via a specially configured ozone-permeable interface constructed of silicone and/or any of its polymers or congeners.

### CROSS REFERENCE TO RELATED APPLICATIONS

This application claims benefit of Provisional Application Serial No. 60/335,735, filed Nov. 2, 2001.

### BACKGROUND OF THE INVENTION

Ozone (O₃) has unique biological and physico-chemical properties which have wide application in various medical fields. Due to its oxidizing capacity, ozone has been shown to be a powerful antibacterial, antiviral, and antifungal agent. As early as the First World War, ozone's antipathogenic properties were used to treat infected wounds, mustard gas burns, and fistulas. Ozone has proven to be a promising approach to the treatment of additional pathogenic infections such as viral infections. Preliminary research on hepatitis C infection has shown that reduction of viral load by means of ozone therapy may significantly normalize hepatic enzymes (SGOT, SGPT, alkaline phosphatase) and improve patient health. In fact, volunteers administered ozone therapy achieved a viral load reduction on the order of 5 log, or 99.999%.

The treatment of blood and other biological fluids or liquids with ozone, for purposes of decontamination from microbial organisms, has presented special challenges. A salient problem centers on properly dosing ozone, which is a gas, into a biological fluid in such a way so as to ensure its homogeneous distribution and minimize damage. Traditionally, ozone is introduced to biological fluids by simply injecting gaseous ozone into the fluid, e.g., bubbling ozone via a syringe or similar device into the fluid to be treated. Often the fluid is agitated as the ozone is introduced. Alternatively, bottles are rotated on rollers so that a thin film of blood lining the inside of the rotating bottle is more evenly exposed to the ozone mixture. For example, a patients blood is collected in a sterile bottle. Subsequently, usually by means of a syringe, a dose of ozone/oxygen gas is added to the blood. U.S. Pat. No. 4,632,980 describes a typical method where ozone is bubbled through the blood. U.S. Pat. No. 4,968,483 describes an alternative method where bottles are rotated on rollers so that a thin film of blood lining the inside of the rotating bottle may more evenly be exposed to ozone. U.S. Pat. No. 5,882,591 describes dispersing blood into fine droplet and exposing the droplets to ozone.

Several disadvantages and drawbacks present themselves in these methods. Injecting a gaseous mixture into blood subjects the blood to foaming which creates physical characteristics making ozone exposure uneven and variable. Even though gas to fluid dynamics apply so that gas tends to be quickly dissipated in its fluid medium, gas bubbles expose higher concentrations of ozone to blood in contact with their surface. Blood located some distance from the bubble surface will experience lessened exposure. Moreover, injecting ozone into blood by means of a syringe increases the incidence of hemolysis through mechanical trauma and denatures proteins at the gas/liquid interface, as do the rolling action of rotating bottles and the dispersion of blood into fine droplets. In addition, none of these methods enable a determination of the amount of ozone actually diffusing into the blood.

Thus, there is a need for a method of interfacing ozone with blood that exposes the blood to minimal mechanical trauma, affords a low hemolysis rate, and allows for an even distribution and homogeneous transfer of the ozone into the blood that is accurately quantifiable.

### BRIEF SUMMARY OF THE INVENTION

An apparatus and method for interfacing ozone containing gaseous mixtures with blood that obviate the prior art difficulties and drawbacks is provided. There is minimal mechanical trauma to cellular elements which translates into a low hemolysis rate. The apposition of gas to blood allows for a transfer and distribution of the gas into the fluid in a uniform, homogeneous, and quantifiable manner. The blood to ozone interface may comprise a membrane, for example in a flat and/or tubular configuration, that allows for the diffusion of a gas mixture quickly, efficiently, and consistently through the interface. Membranes comprising silicone and/or any of its polymers or congeners are suitable for use in this invention. The interface may be coated with an anticoagulant and/or have an anticoagulant agent bound to it.

According to one aspect of the invention, there is provided a method of reducing the viral load in blood by contacting the blood with a sufficient amount of ozone wherein the blood is passed through a cartridge comprising a gas permeable interface, i.e., a gas permeable membrane or a gas permeable tube, where one surface of the interface is exposed to ozone and the opposite surface exposed to blood. The ozone passes through the interface and into the blood at a controlled rate. The path for the blood is sufficiently narrow so that the blood is substantially uniformly exposed to the ozone.

According to another aspect of the invention, there is provided an apparatus for disinfecting biological liquids with ozone containing gas. The apparatus comprises an ozone generator for producing a controlled concentration of ozone and a cartridge comprising a gas permeable membrane and a narrow passage along the membrane for the biological liquid. The invention also provides an apparatus comprising a cartridge which further comprises a gas permeable membrane and one or more narrow passages along the membrane for the biological liquid. Alternatively, or in addition to, the biological liquid may pass through gas permeable tubular membrane held within an atmosphere of gaseous ozone. The path for the biological liquid is sufficiently narrow so that the biological liquid is uniformly exposed to ozone passing through the membrane and into the biological liquid. The path may comprise multiple sub-paths and may be serpentine.

According to a further aspect of the invention, there is provided a method of ozonation of blood and biological fluids by means of diffusion through a bilayer membrane. In one embodiment the bilayered membrane comprises silicone and/or any of its polymers or congeners. Either an ozone containing gas or the biological liquid may flow within the bilayer membrane.

Yet another aspect of the invention provides for precise ozone dosing of the biological fluid by means of a bilayered membrane, a controlled flow rate of the biological fluid, and constant ozone pressure and concentration. A bilayered membrane comprising silicone and/or anyone of its polymers or congeners allows for steady, reliable diffusion of the ozone through the membrane and into the biological fluid. A controlled biological fluid flow rate inside the bilayered membrane is provided by a peristaltic pump. A rigid casing surrounding the interface membrane allows for constant ozone pressure on the outside of the membrane to be maintained. The precision of ozone dosing may be further enhanced by controlling the ozone pressure and flow rate past the exterior of the membrane and within the rigid casing. Alternatively, ozone may be exposed to the interior of the membrane and the biological fluid to the exterior of the membrane and within the casing. The pressure and flow rate may be monitored with a built-in pressure transducer and flow measurement device. The concentration of ozone may be monitored by a UV detector. In addition, the precision of ozone dosing may be further enhanced by an apparatus which allows countercurrent exchange between the gas and liquid phases.

### BRIEF DESCRIPTION OF THE DRAWINGS

FIG. 1 illustrates a schematic circuit diagram of an ozone generator according to one embodiment of the present invention.

FIG. 2A illustrates a schematic circuit diagram of a cartridge having a gas permeable membrane according to one embodiment of the present invention.

FIG. 2B illustrates a side view of the cartridge of FIG. 2A.

FIG. 3 illustrates the relationship of ozone contactor unit (45) to ozone generator (42), peristaltic pump (51), and fluid (50) and (54) undergoing ozonation.

FIG. 4A illustrates a front view of an ozone contactor interface (OCI).

FIG. 4B illustrates a side view of an ozone contactor interface (OCI).

FIG. 5A illustrates a front view of an ozone contactor casing (OCC) in its unfolded configuration.

FIGS. 5B and 5C illustrate an ozone contactor casing (OCC) in its folded position with a back view and a side view, respectively.

FIG. 6 illustrates ozone permeation rates over time of several different membranes.

FIG. 7 illustrates optimization of ozone permeation rates verses fluid flow rate of several different membranes.

### DETAILED DESCRIPTION OF THE INVENTION

According to the invention, methods and delivery systems for contacting a biological liquid with ozone to decrease the amount of pathogenic agents in the biological liquid is provided. Biological liquids include, but are not limited to blood and blood products such as plasma, and serum. The system comprises a medical grade ozone generation portion and a delivery portion which is referred to as a cartridge. The cartridge comprises a gas permeable membrane which allows for a controlled amount of ozone to be delivered to the blood.

The concentration of ozone delivered to a patient must be accurately quantified. Reasons include (1) the fact that ozone in too high concentrations is toxic to whole blood cellular elements and causes hemolysis; (2) that immune system stimulation of cytokines occurs within an optimal range; and (3) that different microorganisms have different susceptibilities to ozone challenge. Ozone, therefore, like many medications, may be said to have a therapeutic window. Below the lower limits of the window, few biological or otherwise therapeutic effects occur, while beyond the window toxic effects are noted. Administration of increasing dosages of ozone to whole blood shows that beyond a certain threshold there is a rise in the rate of hemolysis. This threshold, depending upon various parameters, is reached at between about 40 to about 60 micrograms per milliliters, and becomes significant when higher levels are attained.

Leukocytes show good resistance to ozone because they have enzymes which protect them from oxidative stress. These enzymes include superoxide dismutase, glutathione reductase, and catalase. Research has shown that platelets also maintain their integrity after ozone administration. In ozone viral load reduction therapy, the doses applied to blood do not disrupt its cellular elements. On the contrary, they tend to stimulate leukocyte function. Ozone increases the oxygen saturation (pO₂) in erythrocytes and enhances their pliability so that capillary circulation is facilitated.

In contrast to the relative stability of whole blood to ozone, virions are adversely affected by ozone in a variety of ways. Ozone disrupts envelope proteins, lipoproteins, lipids, and glycoproteins, and the presence of numerous double bonds in these unsaturated molecules makes virions vulnerable to the oxidizing effects of ozone. Double bonds are thus reconfigured, molecular architecture is disrupted and widespread breakage of the envelope ensues. Deprived of an envelope, virions cannot sustain nor replicate themselves.

In addition, the introduction of ozone into the serum portion of whole blood induces the formation of lipid and protein peroxides. While these peroxides are not toxic to the host in quantities produced by ozone therapy, they nevertheless possess oxidizing properties of their own which persist in the bloodstream for several hours. Lipid peroxides created by ozone administration show long-term antiviral effects which serve to further reduce viral load. This factor may explain in part the reason for the fact that ozonated blood in the amount processed in the treatment protocol (50 to 300 milliliters) may reduce the viral load value in the total blood volume (approximately 7 liters).

Ozone treatment provides additional benefits such as inducing the release of cytokines. Cytokines are proteins manufactured by several different types of cells which regulate the functions of other cells. Mostly released by leukocytes, they are important in mobilizing the immune response. Thus, the ozone-induced release of cytokines is a significant means for the reduction of circulating virions.

Ozone action on viral particles in infected blood provides additional indirect benefits. One benefit is the modification of virions so that they are sufficiently dysfunctional so as to be nonpathogenic yet remain grossly structurally intact. This attenuation of viral particle functionality through slight modifications of the viral envelope by ozone eliminates pathogenicity and at the same time provides an immunogen to function as an autovaccine. In view of the fact that so many mutational variants exist in any one afflicted individual, the creation of an antigenic spectrum of crippled virions provides for a unique host-specific stimulation of the immune system, thus designing what may be called a host-specific autovaccine. The viral load inhibition in this scenario offers unique therapeutic specificity in that the attenuated virions are species-specific to the host. The resulting antibodies formed in response to the host-specific virions provides for a therapeutic tactic which conventional vaccines have been unable to attain.

Alternatively, viral integrity may be completely destroyed by the ozone challenge resulting in fragmented circulating virions. These cleaved fragments in turn stimulate the immune system to elaborate antibodies. A great variety of viral fragments are created by this mechanism and the nature of the host immune response is likely to be idiosyncratic.

The ozone generation portion of the system comprises a source of oxygen connected to an ozone generator. Preferably, the generator provides an adjustable and consistent oxygen flow rate which will permit accurate control over the final ozone concentration. The oxygen channeled to the generator is preferably of medical grade purity.

To generate ozone, oxygen is imparted energy in order to split some if its molecules so that single oxygen atoms may then react with diatomic oxygen molecules to form ozone (O₃). Energy may come from different sources such as coronal discharge, ultraviolet radiation and microwave energy. A preferred method of generating ozone is by microwave energy.

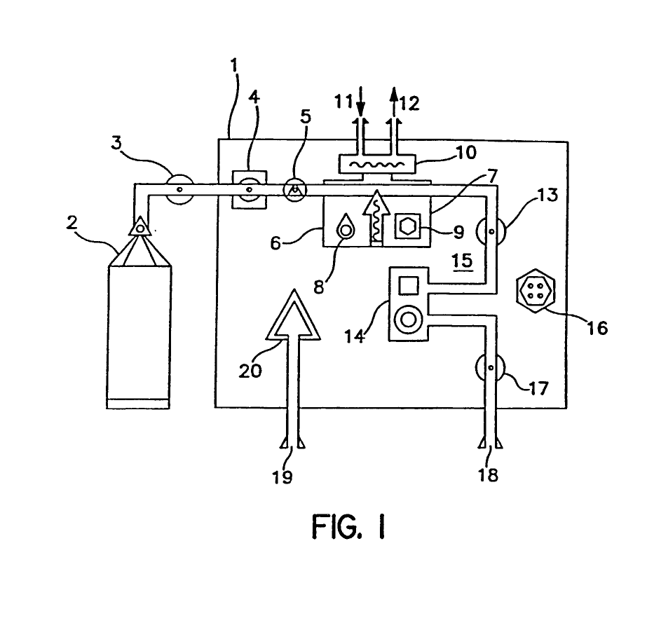

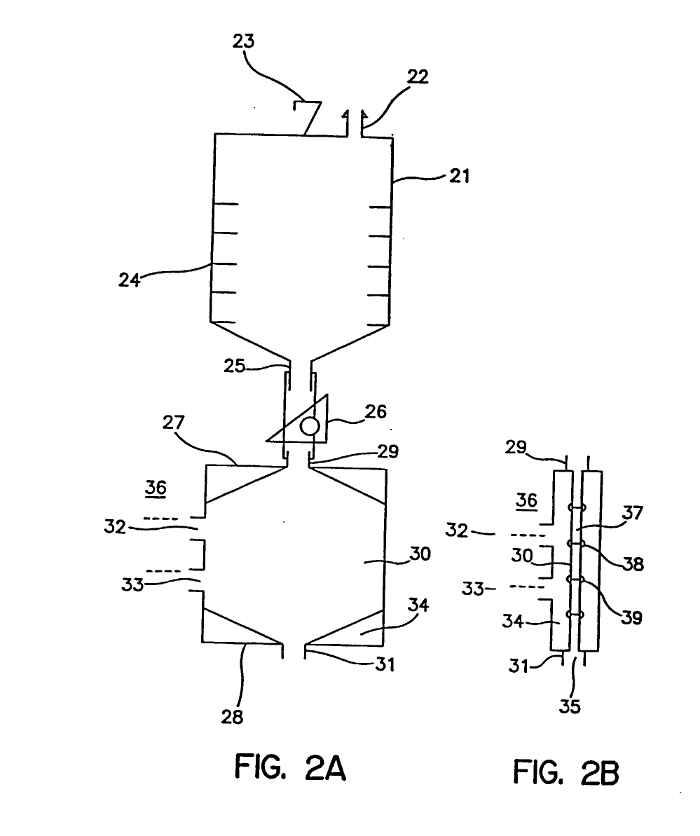

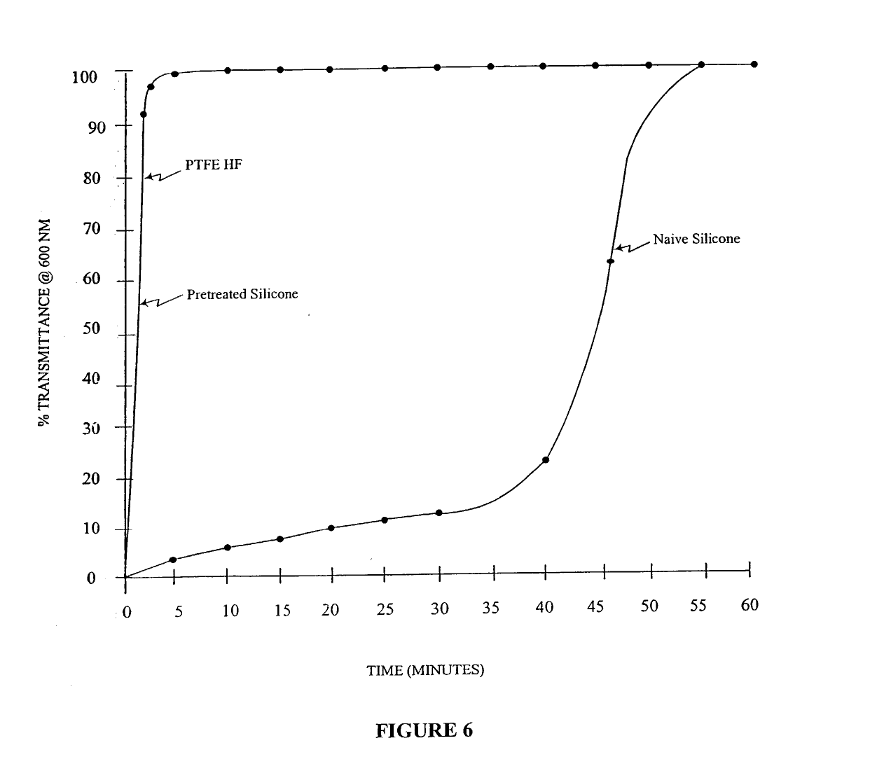

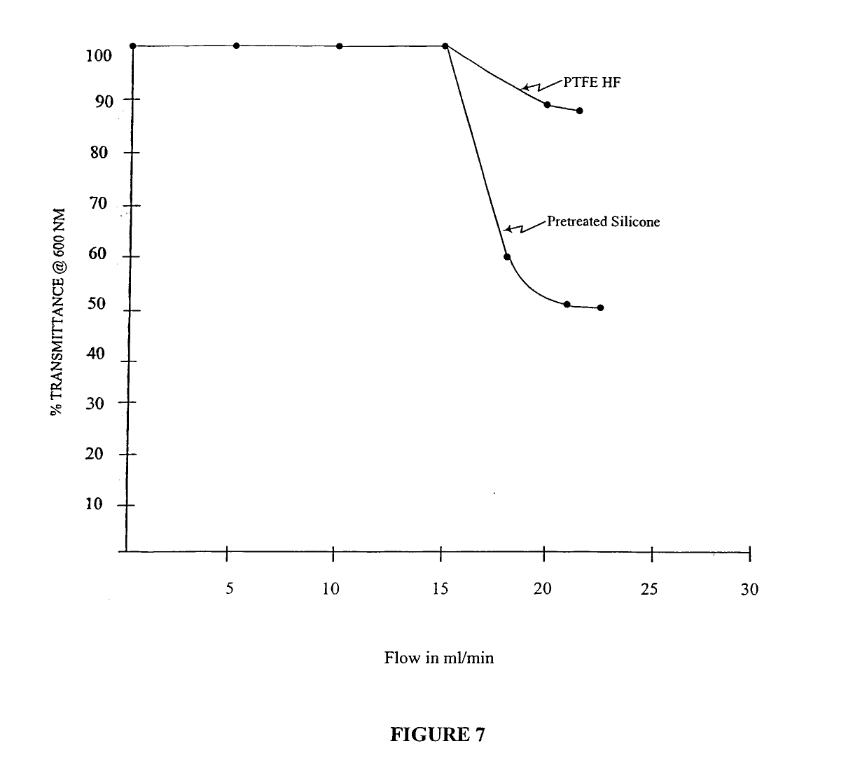

Referring now in detail to the drawings, a biological liquid treatment system in accordance with the invention is illustrated generally in FIGS. 1 through 5. FIG. 6 shows that pre-treated silicone, like PTFE, immediately passes ozone. FIG. 7 illustrates ozone permeation verses flow rate. The biological liquid treatment system comprises an ozone generator (FIG. 1) and a disinfecting gas and biological liquid interface apparatus, or cartridge (FIGS. 2-5). Ozone generator (1) generates and administers a controlled amount of ozone to the biological liquid interface apparatus in order to disinfect the biological liquid.

As shown in FIG. 1, ozone generator (1) receives medical grade oxygen from an oxygen supply tank (2) regulated by control flow (3). The ozone generator (1) comprises a flow rate gauge (4) for measuring the exact oxygen flow rate entering the ozone generator (1), and a flow rate regulator (5) fine tuning the oxygen flow rate.

The conversion of oxygen into ozone is by way of an energy module (6). The energy module (6) utilizes an appropriate electromagnetic discharge, such as, for example, coronal discharge, plasma discharge, UV radiation, or microwaves. An amperage digital gauge (9) measures, in amperes, the energy channeled into the energy module (6), while an amperage regulator (8) modulates energy output such that higher energy levels result in greater concentrations of ozone.

The generator (1) also comprises a cooling element (10). Cooling element (10) includes an intake (11) and an outlet (12). The heat produced by the energy module (6) is dissipated by water entering the intake (11) and exiting the outlet (12) of the cooling element (10).

The generator (1) further comprises a bar pressure gauge (13) for measuring the internal gas pressure of the generator.

The amount of generated ozone is monitored by an ozone analyzer (14) and displayed by an ozone digital gauge (15).

The generator (1) also includes a computer interface port (16) connected to the components of the generator (1). The computer interface port (16) provides an external computer system with data logging that enables a clinician to adjust the treatment parameters of the generator (1), such as, for example, the oxygen flow rate, the internal system pressure, the ozone concentration, the amperage output to the energy module (6), the time functions, and memory functions. In one embodiment, the ozone concentration is monitored continuously at 254 nanometers. Stainless steel pressure transducers are used to determine pressure. The ozone containing gas is dehumidified to increase partial pressure differences across the interface. All equipment parameters are recorded and logged electronically.

An ozone exit flow gauge (17) measures the ozone/oxygen flow rate as they exit the generator through ozone exit port (18). An ozone re-entry port (19) accepts ozone that is returned to the generator for purposes of reversion to oxygen by, for example, an ozone destructor unit (20).

Further information about the operation and construction of ozone generators is provided in U.S. Pat. No. 5,052,382, entitled “Apparatus for the Controlled Generation and Administration of Ozone” to Wainwright.

As illustrated in FIGS. 2A and 2B, the disinfecting gas and biological liquid interface apparatus comprises a collection receptacle (21) and a cartridge (27).

The collection receptacle (21) includes a container having an entry port (22), an exit port (25), and a hook (23). The container contains an anticoagulant, such as, for example, citrate or heparin, and features graduated horizontal markings (24) indicating the volume of blood contained within the receptacle (21). The exit port (25) permits blood to pass through a conduit to a control valve (26). Control valve (26) regulates the blood entering the cartridge (27).

Cartridge (27) includes an interior wall, a liquid flow inlet (29), a liquid flow outlet (31), an ozone inlet (32) and an ozone outlet (33). The ozone inlet (32) and ozone outlet (33) of the cartridge (27) are connected by ozone conduits (36; not shown) to the ozone exit port (18) and the ozone re-entry port (19) of the generator (1).

Cartridge (27) contains a membrane (30). Membrane (30) is impermeable to the biological liquid to be treated but is gas permeable to ozone to permit the diffusion of ozone from the gas mixture quickly, efficiently and consistently into the liquid being treated. The latter requirement is important because several materials used as membranes cannot sustain their molecular integrity in the face of prolonged ozone exposure. Some membranes whose gas diffusion capacity depends upon the presence of micropores are often found to lose efficiency. Electron microscopy shows that micropores exposed to ozone are apt to show loss of patency, probably through oxidation of the polymer molecules lining their lumens, and possibly through the plugging of the micropores by ozonated proteinaceous or lipid material. In one embodiment according to this invention, membranes are made from materials which do not have micropores but nevertheless permit the diffusion of ozone/oxygen through the mesh of their molecular makeup.

Non-limiting examples of membranes having appropriate characteristics for use according to the invention include silicone, polytetrafluoroethylene (PTFE), expanded PTFE, cellulose, polycarbonate, polysulfone, metal, and ceramic membranes. Such membranes are known in the art and described, for example, in Kesting and Fritzsche Polymeric Gas Separation Membranes 1993, John Wiley & Sons, New York and in the Encyclopedia of Chemical Technology, Fourth Ed., Volume 18, 1995, John Wiley & Sons, New York pp. 135-193. One example of a membrane suitable for use according to the invention is a flat-sheet membrane. Other examples include tubular membranes, either circular or with star-shaped cross sections, accordion folded membranes, and other such membrane configurations known in the art. The rate of ozone passage through the interface may be controlled by variation of the membrane thickness. In one embodiment, the interface comprises silicone that is about 100 millimeters to about 50 micrometers thick or comprises silicone that is about 300 micrometers to about 50 micrometers thick. Membranes that are as thin as possible and avoid pinholes in the manufacturing process and tears or ruptures during use are preferred. Such membranes have the highest ozone pass rates.

In one embodiment of the invention, an interface comprising polyethylene hollow fibers was used to deliver ozone to blood. The polyethylene interface was slowly degraded over time by the ozone. Also some protein was denatured and some protein penetrated through the membrane. In one embodiment, an interface comprising PTFE HF (hollow fibers) was used to deliver ozone to blood. PTFE immediately passed ozone, but the liquid pressure needed to be maintained above the gas pressure to avoid bubbles.

In one embodiment of the invention, an interface comprising silicone was used to deliver ozone to blood. Both platinum and peroxide cured silicones passed ozone, and no long-term degradation of either silicone was observed. Maximum passage of ozone through about 1/32 of an inch of silicone was discovered to take place about 30 to about 40 minutes after the initial exposure to ozone. FIG. 6 illustrates the ozone permeation of silicone over time. Naive silicone (approximately 1/32 of an inch thick and 120 cm2) when exposed to ozone (82 g/m3 of ozone in oxygen at 80 inches of water) passes ozone at an increasing rate over time, reaching a maximum passing rate after about 50 minutes. A naive silicone layer that was 200 micrometers thick required about 16-18 minutes to reach maximum permeation. The amount of ozone passing the interface was determined by measuring the change in Indigo Dye concentration at 600 nm. FIG. 6 shows that pre-exposed or pre-treated silicone, like PTFE, immediately passes ozone. FIG. 7 illustrates ozone permeation verses flow rate. Both PTFE and pre-treated silicone, under the conditions described for FIG. 6, exhibit a point at which the flow rate past the interface exceeds the ozone pass rate through the interface. By varying the interface thickness, interface surface area, fluid path length, and/or fluid contact time the desired results in increased permeation rates may be obtained.

It appears that silicone may act as a reservoir for ozone, that silicone and/or impurities in the silicone are reacting with the ozone, and/or that ozone is altering the silicone network. It is likely that the ozone treatment is modifying the silicone network. When pre-treated silicone is re-exposed to ozone, ozone rapidly passes through the membrane within the first few minutes, even months after the initial exposure. Exposing silicone to 80 g/m³ of ozone for three or more hours did not result in a significant loss of strength as determined by both burst test or tensile strength measurements.

A silicone interface of about 0.031 inches was found to rapidly pass ozone. Thinner interfaces will pass ozone more rapidly, thicker interfaces will have greater strength. In one embodiment of the invention, a silicone interface is between about 100 micrometers and about 500 micrometers in thickness, or it is between about 150 micrometers and about 250 micrometers in thickness. The strength of the interface may be increased by casting it on a screen or netting, such as a polyester screen. A silicone interface prevents direct gas-liquid contact due to true ozone molecular diffusion through the silicone interface and, therefore, liquid pressures are not required to be higher than gas pressures to prevent bubbles. Silicone is also anti-thrombogenic, biocompatible, physiologically inert, and its surface charge is similar to the surface charge of blood vessel endothelium. Silicone is easily cast in a variety of shapes, for example, thin films and over screens.

The effect from treating silicone with ozone is non-reversible. The effect is not dependent on the silicon curing agent, as both platinum and peroxide cured silicones show identical results under the same conditions. Gas permeability of silicone is increased by pre-treatment with ozone. In embodiments of the invention, the gas permeability of silicone is increased by about 2 times or more; by about 2 to about 5 times; by about 2 to about 10 times; by about 5 to about 10 times; by greater than about 5 times; or by greater than about 10 times compared to non-pretreated silicone. Elongation and burst tests show there are no significant changes in strength properties of silicone resulting from ozone treatment. Thus, it appears that there is no significant degradation of the silicone structure. Possibly, ozone is reacting with low molecular weight impurities in the silicone matrix, and/or it is altering the silicone matrix in other ways, for example by oxidation or a free-radical process. Silicone rubber or elastomer has a three-dimensional network structure caused by cross-linking of polysiloxane chains. Free-radical reactions, such as peroxide, or platinum-catalyzed reactions are often employed for the formation of the silicone networks. Cross-linking of extrudable and moldable silicone stock is usually done via peroxide-generated free radicals adding to vinyl groups incorporated along the polymer backbone, or, increasingly, by a platinum-catalyzed addition of silane to terminal vinyl groups. See the Encyclopedia of Chemical Technology, Fourth Ed., 1997, John Wiley & Sons and the Encyclopedia of Polymer Science and Technology, 1970, John Wiley & Sons, both incorporated herein in their entirety. Treatment with ozone is likely altering the silicone network by a similar mechanism. In fact, ozone treatment of polysiloxane may be used to create silicone networks, i.e., curing, without the need for peroxide or platinum curing. Ozone curing of silicone provides both highly pure silicone and silicone with a unique network structure.

The novel method of treating silicone with ozone has may applications. For example, it may be used to remove unreacted monomers from silicone to achieve a high-purity silicone for use in medicine or electronics, or the method may be used to create silicone with decreased reactivity. The method may be used to create silicone polymers that have additional cross-linking. In fact, the ozone treatment method may be used to cross-link polysiloxanes, thereby eliminating the need for peroxide or platinum curing. Additional cross-linking may be achieved by formulating silicone to contain additional monomer or polymers that will cross-link with each other and/or the silicone matrix, or by attachment of ozone-polymerizable groups to the polysiloxane backbone. The ozone treatment method may be used to sterilize silicone articles without adversely affecting mechanical properties of the articles.

These modified silicones have altered reactivity making them suitable for a wide variety of applications. The modified silicones may be used for durable medical implants or for making contact lenses. The modified silicones may be used in the fabrication process of electronic components, such as silicon chips. The modified silicones may be used to deliver any gas to any liquid. For example, the modified silicones may be used in blood oxygenation devices. Many of these devices have silicone membranes that diffuse gases at much lower rates than the modified silicones of this invention.

Membrane (30) includes a first side and a second side. The first side of membrane (30) and the interior wall of the cartridge (27) define an area for circulating ozone from the ozone inlet (32) to the first side of the membrane. A portion of the second side of the membrane defines a narrow passageway in communication with the liquid flow inlet (29) and the liquid flow outlet (31). In operation, the ozone passes through the membrane (30) to treat the liquid flowing through the passageway created by a portion of the second side of the membrane (27).

In one embodiment, the cartridge (27) is constructed so that blood flows through one or more narrow passageways defined by a bilayered membrane (37). In other embodiments the one or more passageways are defined by a tube membrane or channels. In yet other embodiments the one or more passageways are defined by a membrane and another surface, such as the interior of the cartridge. The distance separating the bilayers or membrane and other surface or forming the tube cross section needs to be narrow enough so as to permit uniform exposure of blood to ozone. At the same time, the passageway defined by the membrane bilayer, or membrane and other surface, or forming the tube cross section must be large enough to allow for adequate blood flow. In the case of bilayer passageways, i.e., membrane and other surface passageways, or tube cross sections, some embodiments use a passageway thickness of between about 1 centimeter and about 0.001 millimeter. Some use a thickness of between about 0.5 centimeter and about 0.1 millimeter. Others use a thickness of between about 5 millimeter and about 0.15 millimeter. Still others use a thickness of between about 1.5 millimeter and about 0.2 millimeter. The width of the passageway may be of any size. For example, a passageway that is between about 1 centimeter and about 0.001 millimeter thick may be from about 0.5 millimeter to about a meter or more in width. In one embodiment, the passageway is about 1 millimeter to about 0.1 millimeter in thickness and about 0.1 centimeter to about 10 centimeter in width or about 0.5 millimeter to about 0.15 millimeter in thickness and about 0.5 centimeter to about 2 centimeter in width. The thickness of the passageway may be determined under conditions of use, or the thickness of the passageway may be determined in the unused article. If the passageway is partially defined by an interface and partially defined by another surface, the passageway should be thinner than described above.

In one variant, the bilayer is held in its configuration by internal trabeculae (38) and/or by external buttresses (39). In another variant, the bilayer is welded together at various points. The thickness of the passage way may be controlled by a variety of methods. For example, by external pressure from a casing or from a gas. The membrane may be wrapped tightly around itself and held in place with a casing, with gas pressure, or vacuum. Spacing for the liquid or gas flow, either inside or outside the membrane, may be provided by placing a network either inside or outside the membrane, or both. Alternatively, or in addition to, channels may be created in the membrane.

The O₃/O₂ (or any other ozone containing gas mixture) maintained outside the membrane (34) diffuses through the membrane and into the blood at a substantially constant rate. The blood inside the membrane also flows at a substantially constant rate. In this manner, integrating the ozone concentration, the ozone diffusion rate through the membrane, and the blood flow rate through the cartridge, the dosage of ozone administered to the blood easily may be determined and regulated. The passageway may be lengthened or shortened depending on the amount of ozone required. One method for varying passageway length is to vary the tortuousness of the passageway. The passageway may also comprise multiple sub-passageways that also have varying tortuousness. In one embodiment, the passageway is designed so that 100 milliliters of blood may be treated in about 2 to about 4 minutes, the flowing blood layer is about 0.15 millimeter to about 0.5 millimeter thick, the thickness of the interface is about 300 micrometers to about 100 micrometers, and the area of the passageway interface is about 100 to about 500 cm².

The cartridge seen from a frontal perspective (28) (FIG. 2A), and a side view perspective (35) (FIG. 2B), shows a cartridge blood inflow port (29). The ozone entry port (32) accepts the ozone containing gas mixture from the generator which circulates within the cartridge outside its membrane in the cartridge O₃/O₂ Space (34). The ozone exit port (33) returns gas to the generator's destructor unit (20). The cartridge blood exit port (31) channels ozonated/oxygenated blood to the patient.

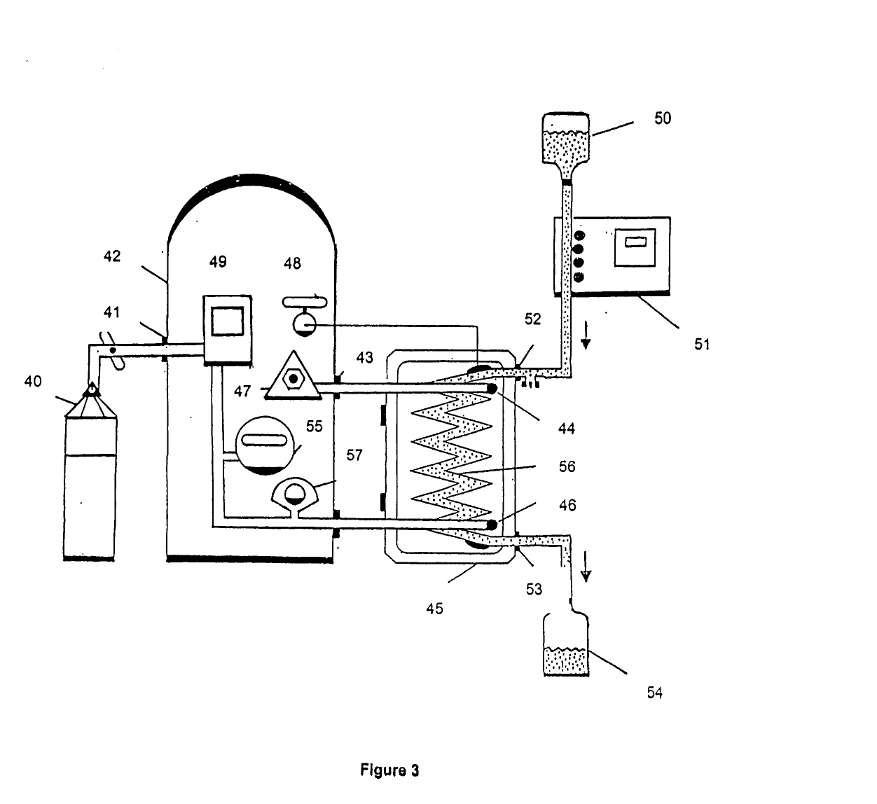

FIG. 3 shows a comprehensive overview of one example variant illustrating principles of the invention. Medical grade oxygen tank (40) feeds ozone generator (42) at its point of intake (41). A precise concentration of ozone to oxygen is measured by ozone analyzer (49). Precise gas flow measurement is provided by flow gauge meter (55). Gas pressure gauge (57) provides for control of the ozone pressure delivered to the system. The ozone/oxygen gas mixture enters ozone contactor module (OCM) (45) at ozone inlet (46). The ozone mixture enters the cavity of the OCM and interfaces with both sides of an ozone permeable membrane or ozone contactor interface (OCI) along a serpentine path (56) which contains a biological fluid or liquid. At this point the biological fluid gets ozonated. The ozone mixture exits OCM (45) at ozone outlet (44) and returns to the ozone generator (42). Unused ozone is returned to its oxygen (O₂) form via ozone destructor (47). A gas connection or fitting (43) allows for quick disconnection of tubing. In one variant, the fluid flow and the ozone flow are countercurrent to each other, although then need not be. Using the counter current variant increases the homogeneity of ozone transfer to the fluid being treated. Transducer (48) measures internal gas pressure in OCM (45). A biological fluid to be ozonated exits from receptacle (50) and flows through peristaltic pump (51) which provides precise flow through the system. The biological fluid enters OCM (45) at inlet (52) and exits OCM (45) at outlet (53) for collection into receptacle (54).

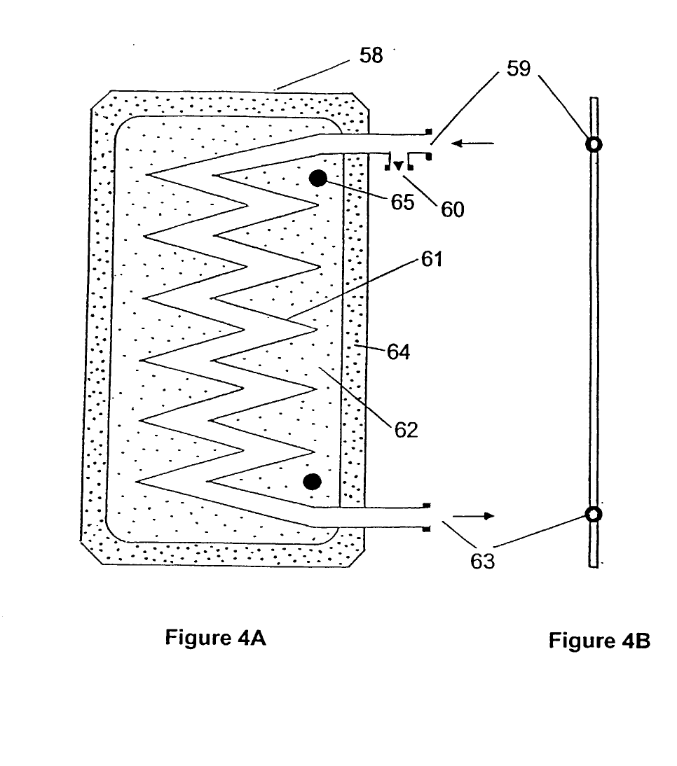

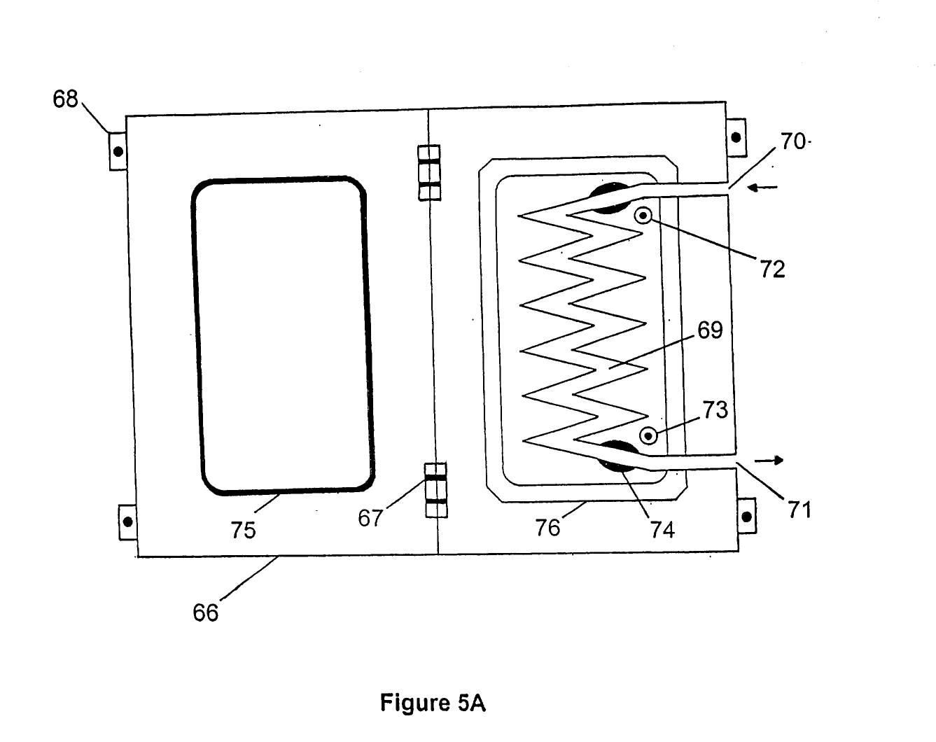

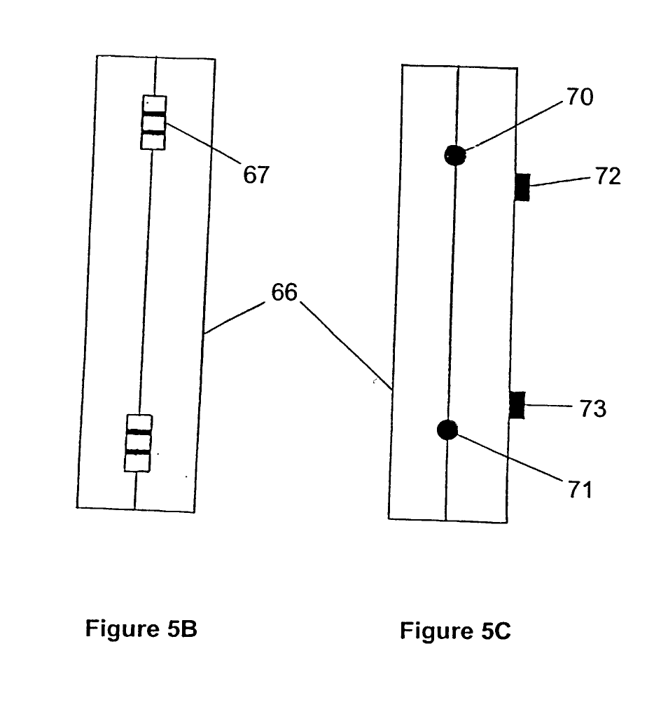

As illustrated in FIGS. 4A-5C, an ozone contactor module (OCM) is functionally made up of two parts, namely ozone contactor interface (OCI) (58) and ozone contactor casing (OCC) (66). By way of example, OCI (58) is a bilayer membrane comprising an interior surface and an exterior surface, a liquid flow inlet (59) and a liquid flow outlet (63). The interior surface of the bilayer membrane defines one or more passageways for a biological fluid passing from liquid flow inlet (59) to liquid flow outlet (63). In some variants, the OCI comprises silicone and/or anyone of its polymers or congeners. As shown, the interface membrane is sufficiently thin to allow rapid ozone diffusion. The interior surface of the OCI defines a narrow serpentine passageway (61) between the liquid flow inlet (59) and liquid flow outlet (63). In other variants, the passageway comprises one or more passageways that are either straight or curved. The biological fluid enters the passageway at liquid flow inlet (59) and exits the passageway at liquid flow outlet (63). The passageway configuration allows for increasing the total surface area of the fluid to be exposed to ozone, while concomitantly ensuring constant fluid flow through the system.

In one embodiment reduced to practice and similar to that shown in FIG. 4A, the outside dimensions of the OCC is 8 by 12 inches and the total length of the serpentine passageway is about 4 feet. The serpentine passageway is between about 5 centimeters and 0.1 centimeter in width and has a working passageway thickness of about 0.15 millimeter to about 0.25 millimeter. Specifically, the serpentine passageway in this embodiment is about 1 centimeter across. Structural support (64), which may be made of stronger, biologically inert material such as polyurethane, may be provided for additional support of the interface. In this example, area (62) depicts welding of two silicone layers to configure the passageway into a serpentine geometry. Opening (60) shows a vent so that bubbles present in the biological fluid may be removed. Communication opening (65) increases the free circulation of ozone to both sides of OCI (58).

As illustrated in FIG. 5A, ozone contactor casing (OCC) (66) surrounds the OCI and comprises two parts held together by hinges (67). OCC (66) includes latches (68) and a seal (75). OCC (66) also includes a template (76) which is in communication with a biological fluid inlet (70) and with a biological fluid outlet (71). OCC (66) further includes ozone inlet (73) and ozone outlet (72) as well as transducer (74). The casing provides for the even distribution of an oxygen/ozone mixture and for its flow through OCM (66) at a given pressure. This assures a steady ozone environment, central to consistent ozone dosing. The casing may be made of plastic, glass, or steel, or any material resistant to ozone oxidation. It is hinged so that, when folded, a hermetic seal provided by seal (75) will be in place. Latches (68) secure the casing in its folded position. The casing allows OCI (58) to fit into its cutout template (76), including a serpentine template configuration (69). Opening (70) accommodates liquid flow inlet (59) of OCI (58) and opening (71) accommodates liquid flow outlet (63) of OCI (58). Opening (73) is the point of ozone entry into the OCC, and opening (72) is the point of exit. Transducer (74) measures the OCC internal gas pressure. FIGS. 5B and 5C show OCC (66) in its rear side view and front side view, respectively.

It should be understood that the OCC need not by configured as shown. For example, the OCC may also be shaped like a canister of cylindrical shape that is configured to accept an OCI through an opening in its side. In one variant, the OCI may be configured in a roll comprising one or more layers. In another variant, the OCI may be a coil of tube or random packing of tube. The specific configurations, i.e. length, width, and thickness, of the passageways are not important as long as flow rates are controllable and results are reproducible.

All publications, patents and articles referred to herein are expressly incorporated herein in toto by reference thereto. The following examples are presented to illustrate the present invention but are in no way to be construed as limitations on the scope of the invention. It will be recognized by those skilled in the art that numerous changes and substitutions may be made without departing from the spirit and purview of the invention.

### EXAMPLES

In this protocol, viral load determines the frequency of treatments and the duration of therapy. Since hepatitis B virus (HBV), hepatitis C virus (HCV), and human immunodeficiency virus (HIV) all show cycles of viremia, this therapy aims at repression of viral expression over long periods of time in order to decrease morbidity and mortality and to increase longevity and quality of life.

A patient is prepared for venipuncture. A volume of blood is withdrawn and is channeled into a sterile collection receptacle containing anticoagulant (citrate or heparin). Depending upon the clinical situation at hand, this volume of blood may range from 50 to 300 milliliters. In the event that the patient's veins are easily accessible, blood may be made to flow directly from the venipuncture intravenous line to the collection receptacle. In situations where veins have poor accessibility a syringe is used. The collection receptacle is constructed of soft transparent plastic. This serves to allow viewing of the blood being treated and, importantly, to minimize cell injury which occurs in hard containers. The collection bag is connected to the cartridge so that blood moves from the collection bag to the cartridge by gravity feed. Alternatively, the collection bag is connected to a peristaltic pump which delivers a constant flow to an OCM. Ozone is produced by the generator at a predetermined concentration and flow rate commensurate with the patient's clinical and laboratory status. Concentrations of ozone used in antimicrobial load reduction therapy may range from about 30 to about 150 micrograms/milliliter. Additional ranges of ozone concentration for antimicrobial load reduction therapy may be from about 50 to about 100 micrograms/milliliter or about 60 to about 85 microgram/milliliter. An additional range of ozone concentration for antimicrobial load reduction therapy, particularly antiviral load reduction therapy, is from about 70 to about 75 microgram/milliliter. Flow rates approximate 0.5 to 1 liter per minute. A conduit from the generator feeds ozone to the cartridge. Another conduit returns unspent ozone to the ozone destructor of the generator so that ozone does not diffuse into the treatment area.

Blood is allowed to flow through the cartridge or OCM. Volumes of blood used in antimicrobial load reduction therapy may range from about 10 milliliters to about 1,000 milliliters. Additional ranges of blood volumes for treatment are about 25 milliliters to about 500 milliliters, or about 50 milliliters to about 200 milliliters. An additional range of blood volume for treatment, especially for antiviral load reduction therapy, is about 75 to about 125 milliliters or about 100 milliliters. When the blood flows through the cartridge or OCM it becomes ozonated.

In one embodiment of the invention, the flow rate of the blood through the cartridge or OCM is determined over a period of time, thereby defining the total volume of blood treated. The concentration of ozone in the carrier gas is determined and the volume of ozone containing gas that enters the cartridge or OCM and the volume that exits the cartridge or OCM over a period of time are also determined. Determining the difference in volumes and the concentration of ozone provides the exact amount of ozone delivered to the volume of blood.

In another embodiment of the invention, the difference in concentration of ozone in the carrier gas entering the cartridge or OCM and the concentration exiting the cartridge or OCM as well as the flow rate of the carrier gas is determined. Since ozone is consumed as it enters the blood, the change in ozone concentration may be used to accurately determine the amount of ozone being administered to blood. Typically, the amount of oxygen that is absorbed by the blood need not be calculated to accurately determine the amount of ozone being administered to the blood in this embodiment.

Alternatively, the blood may be pre-saturated with oxygen, if necessary, to obtain a more accurate result. The flow rate of the blood through the cartridge or OCM is determined over a period of time, thereby defining the total volume of blood treated. Factors that may be varied to control the amount of ozone administered to the blood include the ozone concentration in the cartridge or OCM, the pressure of the ozone containing gas in the cartridge or OCM, the ozone permeability of the membrane, the flow rate of the blood, the contact time of the blood with the interface, the flow rate of the ozone containing gas, and the temperature. In one embodiment, the biological fluid and ozone flow countercurrent to each other. In one embodiment, the temperature is 37° C. Since the blood outflow from the cartridge or OCM may be directly connected to the same intravenous conduit used to withdraw blood from the patient, ozonated/oxygenated blood is accordingly returned to the patient via this same route.

One skilled in the art, normally a physician, may optimize the treatment protocol based on the patient and the infecting agent. In one embodiment, a patient may receive treatment every day for a period of about 10 to about 50 days, or a period of about 20 to about 30 days. In another embodiment, a patient receives treatment every 2 to about 4 days for an average number of sessions approximating 5 to about 25, or about 10 to about 15. It is important to note, however, that the treatment protocol is predicated upon sequential clinical and laboratory determinations. Laboratory measures include a comprehensive blood screen including hepatic enzyme quantification, and/or microbial load determinations by polymerase chain reaction (PCR) or other nucleic acid assay known in the art. In the event that microbial load reduction is judged to be satisfactory, treatment may be halted after fewer than the number of indicated sessions and the patient monitored thereafter at regular intervals. If necessary, the treatment period may be extended or repeated. The health, age, weight and sex of a patient may affect the results of the treatment. For example, patients addicted to alcohol or drugs responded less well to treatment than did non-addicted patients. Other diseases, such as hormone imbalance, have also negatively affected patients' responses to therapy.

It is to be understood that the embodiments and variations shown and described herein are merely illustrative of the principles of the present invention. Therefore, various adaptations and modifications may be implemented by those skilled in the art without departing from the spirit and scope of the present invention.

What is claimed is:

1. A method of reducing microbial load in a biological liquid by treating the liquid with gas comprising ozone, wherein the method comprises the steps of:
(a) passing the biological liquid comprising a microbe along at least a portion of a first surface of gas permeable interface, wherein the interface comprises one or more tortuous pathways for the biological liquid, and
(b) contacting a second surface of the gas permeable interface with the ozone for a time and under conditions sufficient to inactivate the microbe in the biological liquid,
thereby reducing microbial load in a biological liquid.

2. The method according to claim 1, wherein the gas permeable interface is contained in a cartridge or module.

3. The method according to claim 1, wherein the interface is a bilayer silicone membrane.

4. The method according to claim 1, wherein the membrane is an ozone-cured silicone membrane.

5. The method according to claim 1, wherein the bilayer membrane comprises a serpentine passageway for the biological liquid.

6. The method according to claim 1, wherein the liquid passes through a passageway, and wherein the passageway has a thickness of between about 1 centimeter and about 0.001 millimeter.

7. The method according to claim 6, wherein the passageway thickness is between about 1 millimeter and about 0.1 millimeter.

8. The method according to claim 7, wherein the passageway thickness is between about 0.15 millimeter and about 0.25 millimeter.

9. The method according to claim 1, where in the biological liquid is blood or a blood product, and wherein the microbe is a virus.

10. The method according to claim 9, wherein the microbe is selected from the group consisting of: hepatitis B virus, hepatitis C virus, and human immunodeficiency virus.

11. The method according to claim 1, wherein the ozone flows counter to a direction of flow of the biological liquid.

12. A method of decreasing viral contamination comprising:
(a) ozonating a virally contaminated biological liquid by passing the biological liquid along a tortuous passageway defined by one side of an ozone permeable material having a first side and a second side, the passageway having a surface area, the surface area having a portion that provides for a substantially uniform exposure of the biological liquid within the passageway to ozone diffusing through the ozone permeable material; and
(b) exposing the portion to a gas mixture containing ozone by contacting the gas mixture with the side opposite the first side.

13. A method of determining an amount of ozone administered to a volume of biological liquid comprising:
(a) exposing a first side of an ozone permeable and biological liquid impermeable interface to a specific volume of a gas comprising a known quantity of ozone;
(b) exposing a second side of the interface to a specific volume of biological liquid; and
(c) measuring a decrease in volume of the ozone containing gas, thereby determining the amount of ozone administered to the volume of biological liquid.

14. The method according to claim 13, wherein the interface is an ozone-cured silicone membrane.

15. An apparatus for disinfecting a biological liquid by contact with ozone, the apparatus comprising:
a cartridge or module including an interior wall, an inlet through which the liquid may enter the cartridge or module, an outlet through which the liquid may exit the cartridge or module, an inlet through which a gas comprising ozone may enter the cartridge or module, an outlet through which the gas comprising ozone may exit the cartridge or module;
a gas permeable membrane contained within the cartridge or module, a first side of the membrane and an interior wall of the cartridge or module defining an area allowing ozone from the ozone inlet to contact the first side of the membrane, a second side of the membrane having a portion defining a passageway coupled to both the liquid flow inlet and the liquid flow outlet so that ozone introduced to the first side diffuses through the portion into the passageway and disinfects the biological liquid flowing through the passageway.

16. An apparatus for disinfecting a biological liquid by contact with ozone, the apparatus comprising:
an ozone contactor casing comprising an interior wall, a liquid flow inlet, a liquid flow outlet, an ozone inlet and an ozone outlet; and
a bilayer membrane having an inside and an outside and contained within the casing, the outside of the membrane and the interior wall of the cartridge defining a volume for circulating gas comprising ozone from the ozone inlet to a section of the membrane, the section defining a passageway in communication with the liquid flow inlet and the liquid flow outlet, the portion being ozone permeable but impermeable to the liquid so that ozone diffusing through the membrane into the passageway will disinfect the liquid flowing through the passageway.

17. The apparatus according to claim 16, wherein the membrane is a silicone membrane.

18. The apparatus according to claim 16, wherein the membrane comprises a serpentine passageway for the biological liquid.

19. The apparatus according to claim 16, wherein the passageway is between about 1 centimeter and about 0.001 millimeter in thickness.

20. The apparatus according to claim 19, wherein the passageway thickness is between about 1 millimeter and about 0.1 millimeter.

21. The apparatus according to claim 20, wherein the passageway thickness is between about 0.15 millimeter and about 0.25 millimeter.

22. The apparatus according to claim 16, wherein the ozone flows counter to a direction of flow of the biological liquid.

23. An apparatus comprising:
a gas inlet;
a gas outlet;
a volume connecting the gas inlet with the gas outlet;
a biological liquid inlet;
a biological liquid outlet;
a biological liquid volume defining a tortuous path between the biological liquid inlet and the biological liquid outlet; and
a membrane dividing the volume from the biological liquid volume, at least a portion of the membrane being ozone permeable but biological liquid impermeable so that ozone in a gas introduced into the volume and contacting the membrane will diffuse through the membrane and into the biological liquid.

24. The apparatus according to claim 23, wherein the path comprises multiple sub-paths.

25. The apparatus according to claim 24, wherein the path is serpentine.

26. A disinfection system comprising:
an ozone generator;
a computer, coupled to the ozone generator;
a biological liquid receptacle;
a control valve, coupled to the biological liquid receptacle, configured to adjustably regulate a flow of biological liquid out of the biological liquid receptacle; and
a cartridge or module, coupled to both the ozone generator and the biological liquid receptacle, configured to receive an ozone containing gas and a biological liquid, the cartridge including an ozone permeable membrane separating the gas from the biological liquid while allowing at least some of the ozone to diffuse through the membrane into the biological liquid under treatment parameters controlled by the computer.

27. A method for curing silicone comprising treating silicone with ozone.

28. The method according to claim 27, wherein the silicone is precured with an agent other than ozone prior to curing with ozone.

29. The method according to claim 27, wherein the silicone comprises polysiloxane.

30. The method according to claim 27, wherein the silicone consists essentially of polysiloxane.

31. An article comprising silicone wherein the article is cured with ozone.

32. The article according to claim 31, wherein the article is precured with an agent other than ozone prior to curing with ozone.

33. The article according to claim 31, wherein the article has greater gas permeability than an analogous non-ozone cured silicone article.

34. The article according to claim 31, wherein the article has greater ozone permeability than an analogous non-ozone cured silicone article.

35. The article according to claim 31, wherein the article has greater oxygen permeability than an analogous non-ozone cured silicone article.

36. The article according to claim 35 for use in a blood oxygenation device.

37. A method of determining an amount of gas administered to a volume of a biological liquid comprising:
(a) exposing a first side of an ozone-cured silicone interface to a specific volume of a gas;
(b) exposing a second side of the interface to a specific volume of a biological liquid; and
(c) measuring a decrease in the volume of gas.

38. The method according to claim 37, wherein the biological liquid is blood.

39. The method according to claim 38, wherein the gas is oxygen.
